(19) United States
(12) Patent Application Publication
(10) Pub. No.: US 2022/0016119 A1
(43) Pub. Date: Jan. 20, 2022

(54) METHODS AND TREATMENTS FOR ERECTILE DYSFUNCTION

(71) Applicant: Record Buck Farms Medical Cannabis, LLC, Howey in the Hills, FL (US)

(72) Inventor: John V. Rivera, Vero Beach, FL (US)

(73) Assignee: Record Buck Farms Medical Cannabis, LLC, Howey in the Hills, FL (US)

(21) Appl. No.: 17/309,098

(22) PCT Filed: Oct. 23, 2019
(86) PCT No.: PCT/US2019/057557

§ 371 Date: Apr. 23, 2021

Related U.S. Application Data
(60) Provisional application No. 62/749,247, filed on Oct. 23, 2018.

(51) Int. Cl. A61K 31/519; A61K 31/4985; A61P 15/10

(52) U.S. Cl. CPC A61K 31/519; A61P 15/10; A61K 31/4985

(57) ABSTRACT

In one embodiment, a pharmaceutical composition for treatment of erectile dysfunction is provided including a therapeutically effective amount of a phosphodiesterase-5 (PDE-5) inhibitor, or a pharmaceutically acceptable salt thereof, and a therapeutically effective amount of at least one of tetrahydrocannabinol (THC), and cannabidiol (CBD).

### BACKGROUND

The endo-cannabinoid system is a central regulatory system that affects a wide range of biological processes in our body. It consists of a group of molecules known as cannabinoids as well as the cannabinoid receptors that they bind to. The cannabinoids are involved in the control of a variety of physiological processes including appetite, pain-sensation, mood, motor learning skills and memory. The human body has two receptors that cannabinoids will bind to, CB-1 receptors in the brain and CB-2 receptors in the body and immune system. Research shows cannabinoid receptors are found in the nervous system, cardiovascular system, reproductive system, the urinary tract and gastrointestinal tract. THC tends to bind to the CB-1 receptors in the brain, creating that “heady” high, whereas CBD is drawn to the CB-2 receptors in the body, giving the patient more of the “body” buzz (non high). In non-technical language, the endo-cannabinoid system is simply a bridge between body and mind and its job is to maintain internal harmony.

Marijuana is the source of over 120 known cannabinoids, including THC and CBD, but the human body also produces several of its own cannabinoids. Our bodies naturally produce endo-cannabinoids to maintain a healthy system. When we are ill, the manufacturing of endo-cannabinoids decreases. Intake of additional cannabinoids which imitate natural endo-cannabinoids and use the same receptors, helps to increase an internal stability again and helps us heal faster. Cannabinoid combinations are unique to each plant and are responsible for provide in the natural symptom relief, that so many sick people are searching for.

Sildenafil® (is a medication used to treat erectile dysfunction, an anti-erectile dysfunction drug). The primary indication of Sildenafil is treatment of erectile dysfunction (inability to sustain a satisfactory erection to complete intercourse). Its use is now one of the standard treatments for erectile dysfunction. Sildenafil has been a common treatment for erectile dysfunction.

In one study, a 25-mg dose was shown to cause no significant change in erectile quality but did reduce the postejaculatory refractory time. Sildenafil works by inhibiting an enzyme in the body, phosphodiesterase 5 (PDE-5). Sildenafil is one example of a PDE-5 inhibitor which causes the blood vessels to relax, increasing blood flow to the penis. As a result, the inhibition of PDE-5 helps to maintain an erection.

Cannabis indica, formally known as Cannabis sativa forma indica, is an annual plant in the Cannabaceae family. It is a putative species of the genus Cannabis. Cannabis indica is used for many purposes such as cloth from the fibers, paper from its bio mass, medical uses, and as a drug source. Cannabis indica produces large amounts of tetrahydrocannabinol (THC). The large amounts of THC found in Cannabis indica results in its popularity for use as a drug for recreational or medicinal purposes. Cannabis strains with CBD:THC ratios above 5:2 are likely to be more relaxing and produce less anxiety than others. This may be due to CBD's antagonistic effects at the cannabinoid receptors, compared to THC's partial agonist effect. CBD is also a 5-HT receptor (serotonin) agonist, which may also contribute to an anxiolytic-content effect. The effects of sativa are well known for its cerebral high. Users can expect a more vivid and uplifting high, while indica is well known for its sedative effects which some prefer for night time use. Indica possesses a more calming, soothing, and numbing experience which can be used to relax or relieve pain. Both types are used as medical cannabis. THC and CBD can be dispensed in a variety of ratios of a combined dose.

THCa is the most abundant cannabinoid in cannabis. THCa is the precursor to THC. Immediately following harvest, THCa begins to convert into THC. THCa is non-psychoactive and has shown to relieve pain, suppress muscle spasms, because it is such a powerful anti-inflammatory. THCa has been found to improve immune system functions as well. CBD is the principal, non-psychoactive cannabinoid present in the cannabis plant and occurs, second in concentration to only THC. CBD is a powerful anti-seizure medication and has been shown to be a better anti-inflammatory than Ibuprofen. CBD can be as effective as THC for treating pain, reducing nausea, insomnia, clearing up psoriasis and managing tumors. Studies have shown CBD can also be an effective way to relieve anxiety and depression. New studies have shown that CBD can lower blood sugar in diabetics. High blood sugar is one leading cause of erectile dysfunction. CBD alone, however, does not work for everyone. Some seizure disorders, muscle spasms or chronic pain can only be treated with a combination of both CBD and THC.

### SUMMARY

In one embodiment, a pharmaceutical composition for treatment of erectile dysfunction is provided including a therapeutically effective amount of a phosphodiesterase-5 (PDE-5) inhibitor, or a pharmaceutically acceptable salt thereof, and a therapeutically effective amount of at least one of tetrahydrocannabinol (THC), and cannabidiol (CBD).

In one embodiment a method for treating or preventing erectile dysfunction in a patient may be provided. The method may include administering to the patient a therapeutically effective amount of a phosphodiesterase-5 (PDE-5) inhibitor, or a pharmaceutically acceptable salt thereof; and a therapeutically effective amount of at least one of: tetrahydrocannabinol (THC), and cannabidiol (CBD).

In another embodiment, a method of eliciting an erection in a patient is provided. The method includes administering to the patient a therapeutically effective amount of a phosphodiesterase-5 (PDE-5) inhibitor, or a pharmaceutically acceptable salt thereof, and co-administering a therapeutically effective amount of at least one of tetrahydrocannabinol (THC), and cannabidiol (CBD).

### DETAILED DESCRIPTION

### Definitions

As used herein, the terms “subject”, “user” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human of the male gender.

The term “an effective amount” means an amount of an agonist or antagonist satiety factor or compositions comprising thereof that when, administered to a subject for treating a condition is sufficient to effect such treatment for the condition. An effective amount can vary depending on, inter alia, the satiety factor used, the condition and its severity and the age, weight, etc. of the subject to be treated. Sample dosage amounts are described in more detail herein.

“Preventing” or “prevention” refers to a reduction in the risk of acquiring a disease or disorder (i.e., causing at least one of the clinical symptoms of the disease not to develop in a subject that may be exposed to or predisposed to the disease but does not yet experience or display symptoms of the disease). Preferably, prevention refers to the use of a compound or composition in a subject not yet affected by the disease or disorder or not yet exhibiting a symptom of the disease or disorder, for instance a subject not yet exhibiting the symptoms of erectile dysfunction.

“Treating” or “treatment” of any disease or disorder refers, in one embodiment, to ameliorating the disease, disorder, or condition (i.e., arresting or reducing the development of the disease or condition, or at least one of the clinical symptoms thereof) that exists in a subject. In another embodiment, “treating” or “treatment” refers to ameliorating at least one physical parameter, which may be indiscernible by the subject. In one particular example, one physical parameter may include insufficient length of time of an erection, in another example, a physical parameter may include a delayed onset or lack of onset of an erection. In yet another embodiment, “treating” or “treatment” refers to modulating the condition, either physically (e.g., stabilization of a discernible symptom) or physiologically (e.g., stabilization of a physical parameter) or both. In yet another embodiment, “treating” or “treatment” refers to enhancing or initiating an erection in a patient suffering from erectile dysfunction.

### Dosage

The dose administered to an animal, particularly a human, more particularly human of the male gender, in accordance with the present invention should be sufficient to effect the desired response in the animal over a reasonable time frame. One skilled in the art will recognize that dosage will depend upon a variety of factors, including the strength of the particular compositions employed, the age, species, condition, and body weight of the animal. The size of the dose also will be determined by the route, timing and frequency of administration as well as the existence, nature, and extent of any adverse side effects that might accompany the administration of a particular composition and the desired physiological effect. It will be appreciated by one of ordinary skill in the art that various conditions or desired results, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached.

The amount of the compound of the invention administered per dose or the total amount administered per day may be predetermined or it may be determined on an individual patient basis by taking into consideration numerous factors, including the nature and severity of the patient's condition, the condition being treated, the age, weight, and general health of the patient, the tolerance of the patient to the compound, the route of administration, pharmacological considerations such as the activity, efficacy, pharmacokinetics and toxicology profiles of the compound and any secondary agents being administered, and the like. Patients undergoing such treatment will typically be monitored on a routine basis to determine the effectiveness of therapy. Continuous monitoring by the physician will ensure that the optimal amount of the compound of the invention will be administered at any given time, as well as facilitating the determination of the duration of treatment. This is of particular value when secondary agents are also being administered, as their selection, dosage, and duration of therapy may also require adjustment. In this way, the treatment regimen and dosing schedule can be adjusted over the course of therapy so that the lowest amount of compound that exhibits the desired effectiveness is administered and, further, that administration is continued only so long as is necessary to successfully achieve the optimum effect.

### Pharmaceutical Compositions

Various embodiments of the invention are foreseen to have valuable application as constituents of pharmaceutical preparations to treat various conditions generally defined as pathologies. Accordingly, embodiments of the invention also comprise pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50, 100 or 1000, up to 2500 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol and cellulose acetate. The compositions may be contained in a vial, sponge, syringe, tube, or other suitable container.

As used herein, the term “administering” or “administration” includes but is not limited to oral or intravenous administration by liquid, capsule, tablet, or spray. Administration may be by injection, whether intramuscular, intravenous, intraperitoneal or by any parenteral route. Parenteral administration can be by bolus injection or by continuous infusion. Formulations for injection may be presented in unit dosage form, for example, in ampoules or in multi-dose containers with an added preservative. The compositions may take the form of suspensions, solutions or emulsions in oily or aqueous vehicles and may contain formulatory agents such as suspending, stabilizing and/or dispersing agents. Alternatively, the compositions may be in powder form (e.g., lyophilized) for constitution with a suitable vehicle, for example sterile pyrogen-free water, before use. Compositions may be delivered to a subject by inhalation by any presently known suitable technique including a pressurized aerosol spray, where the dosage unit may be controlled using a valve to deliver a metered amount.

Administration by capsule and cartridges containing powder mix of the composition can be used in an inhaler or insufflator to deliver the particles to the female subject. Still other routes of administration which may be used include buccal, urethral, vaginal, or rectal administration, topical administration in a cream, lotion, salve, emulsion, or other fluid may also be used.

The term “co-administration” or “co-administering” as used herein refer to the administration of a substance before, concurrently, or after the administration of another substance such that the biological effects of either substance synergistically overlap. The combination of agents as taught herein can act synergistically to treat or prevent the conditions described herein. Using this approach, one may be able to achieve therapeutic efficacy with lower dosages of each agent, thus reducing the potential for adverse side effects. Using this approach, one may also, or alternatively, be able to achieve improved or enhanced results, not achievable with any of the agents when given independently.

It is noted that the term “pharmaceutically acceptable salt(s)” refers to salts derived from treating a compound of the composition with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids.

CBD and THC are known to decrease anxiety and depression as described in Cuttler, Carrie, et. Al., Journal of Affective Disorders. Vol. 235. Pages 198-205, Elsevier Science, 2018, which is hereby incorporated by reference in its entirety. The safety of cannabidiol has been shown in several clinical studies performed in patient suffering from psychological or neurological disorders. The reports indicate that administration of Cannabidiol in doses of up to 1500 mg/day resulted in no significant adverse effects in patients. In some examples, the doses of cannabidiol or a functional derivative thereof is 50-2500 mg. In other embodiments, the dosage may include between 0.1-2500 mg. In further embodiments, the dosage of cannabidiol may include between 0.1-1000 mg CBD.

Combinations of CBD and THC have been discussed for reducing appetite in patients and contributing to weight loss. Various combinations of CBD and forms of THC have been linked to decrease in rates of diabetes, have been linked to low BMI and decreases in other metabolic diseases. The inventors herein have identified the use of CBD and THC in supporting healthy sexual function in men. In particular, the combination as described herein has been used to reduce or eliminate erectile dysfunction issues in men. See Jadoon K A, Ratcliffe S H, Barrett D A, et al., Diabetes Care. 2016 October; 39(10): 1777-86, incorporated by reference in its entirety herein. Jadoon et al included a randomized, double blind placebo-controlled study of type 2 diabetic, non-insulin treated subjects treated with CBD, THCV at various rations over 13 weeks. THCV and CBD was found to represent a new therapeutic agent in glycemic control in diabetic subjects.

Chronic cannabis use has been shown to cause downregulation of CB1 receptors in areas such as the prefrontal cortex, anterior cingulate cortex, hippocampus, and parahippocampal gyrus (Hirvonen et al., 2012), which are known to be implicated in mood and emotionality (see Drevets et al., 2008 for review); therefore, regular use of cannabis may have longer-term effects on negative affect (that may be reversed following a period of abstinence). See Cuttler et al., 2016.

In one embodiment, a pharmaceutical composition for treatment of erectile dysfunction is provided including a therapeutically effective amount of a phosphodiesterase-5 (PDE-5) inhibitor, or a pharmaceutically acceptable salt thereof, and a therapeutically effective amount of at least one of tetrahydrocannabinol (THC), and cannabidiol (CBD). Moreover, studies have shown that cannabis is a vascular dilator and assists in maintaining consistent blood pressure by keeping blood vessels open, in addition to its effect sin relieving physical pain, anxiety and depression. Inventors have identified that CVB in conjunction with PDE5 inhibitor (e.g. sildenafil) results in a synergistic effect in treating erectile dysfunction.

Studies have identified cannabis as being used for treatment peripheral vascular disease (PVD) as its bioactive agents bind to CB02 receptors on immune cells causing an anti-inflammatory response. See Adejumo, Adeyinka et al. Circulation: Cardiovascular Quality and Outcomes, Vol. 10, Issue Suppl 3. March 2017, which is hereby incorporated by reference in its entirety.

PDE5 inhibitors may be used to treat erectile dysfunction as disclosed in U.S. Patent Application Publication No. 2003/0144296. PDE5 converts cyclic guanosine monophosphate (cGMP) and cyclic adenosine monophosphate (cAMP) to monophosphate. cGMP is a major intracellular effector of smooth muscle relaxation, facilitates increased blood flow, and elicits erection. By reducing the breakdown of cGMP, PDE5 prolongs action of cGMP. Vardenafil (Nuivi, Bayer/GSK) and Tadalafil (Clalis, Lilly ICOS LLC) are two PDE5 inhibitors that may be administered by the methods of the present invention. Vardenafil has been administered orally^{, from} 5 to 20 mg, with onset of the therapeutic effect occurring at about 40 minutes after administration and lasting for about 4 hours. Tadalafil has been administered orally from 10 to 20 mg, with onset of the therapeutic effect occurring at about 16 minutes after administration and lasting up to about 36 hours. for example. However, it has been found that often PDE5 inhibitors alone are not as efficacious as initially thought. In particular, PDE5 inhibitors do not solve the problem of reducing anxiety or pain in the user so as to enhance the sexual experience.

PDE5 inhibitors are described herein; however, the compositions and methods are not limited to use with PDE5 inhibitors. In some non-limiting embodiments other drugs may be used. including, for example, melanocortin receptors, oxytocin and oxytocin receptor agonists, inhibitors of a neuropeptide Y (NPY), dopamine receptor agonists (e.g., apomorphine), melanocortin receptor agonists, intracavernous therapies, growth hormone-releasing peptide receptor agonists, 5-hydroxytryptamine receptor agonists, alpha-adrenoceptor antagonists, topical therapies, guanylyl cyclase activators, and rho-kinase antagonists.

In one example embodiment described herein, the PDE-5 inhibitor may include sildenafil and/or tadalafil. While PDE5 inhibitors are known for treatment of erectile dysfunction, it has been found that often PDE5 inhibitors alone are not as efficacious or are accompanied by unwanted side effects. For example, common side effects of erectile dysfunction medication may include headache, flushing, upset stomach, muscle pain, back pain, and nausea. Additionally, in particular, PDE5 inhibitors do not solve the problem of reducing anxiety in the user so as to enhance the sexual experience. Consequently, as described herein, a synergistic effect of a combination of PDE5 inhibitor and CBD or THC or a combination of both CBD and THC with PDE5 inhibitor may be used for ED side effect treatment.

In one embodiment, the CBD may be extracted from cannabis indica. In another embodiment, the composition may include both THC and CBD, wherein the THC and CBD are provided in a 5:2 ratio, for example.

In yet another embodiment described herein, the composition may include a pharmaceutically acceptable carrier. The pharmaceutically acceptable carrier may include a tablet, a pill, or a capsule, in some non-limiting embodiments.

In another embodiment, the composition may include between 0.01 mg-150 mg of the PDE-5 inhibitor. In yet another embodiment, the composition may include 0.1 mg-100 mg of the PDE-5 inhibitor. In one embodiment, the composition may include 0.01 mg-150 mg of THC. In another embodiment, the composition may include 0.1 mg-100 mg of THC. In another embodiment, the composition may include 0.01 mg-1500 mg of CBD. In still another embodiment, the composition may include 0.1-1000 mg of CBD.

In one embodiment a method for treating or preventing erectile dysfunction in a patient may be provided. The method may include administering to the patient a therapeutically effective amount of a phosphodiesterase-5 (PDE-5) inhibitor, or a pharmaceutically acceptable salt thereof; and a therapeutically effective amount of at least one of: tetrahydrocannabinol (THC), and cannabidiol (CBD). In one embodiment, the PDE-5 inhibitor comprises sildenafil and/or tadalafil. In another embodiment, the method is provided, wherein the CBD is extracted from cannabis indica. In yet another embodiment, the method is provided wherein both THC and CBD are administered to the patient. In one example, the THC and CBD are administered to the patient in a 5:2 ratio, wherein 5 parts THC to 2 parts CBD are administered. In other embodiments, THC and CBD are administered in a 1:1 ratio. In another embodiment, the method is provided wherein the PDE-5 inhibitor is co-administered with THC, or CBD, or with a combination of both THC and CBD.

In another embodiment, a method of eliciting an erection in a patient is provided. The method includes administering to the patient a therapeutically effective amount of a phosphodiesterase-5 (PDE-5) inhibitor, or a pharmaceutically acceptable salt thereof, and co-administering a therapeutically effective amount of at least one of tetrahydrocannabinol (THC), and cannabidiol (CBD).

In still another embodiment, a method of prolonging an erection in a patient is provided, including administering to the patient a therapeutically effective amount of a phosphodiesterase-5 (PDE-5) inhibitor, or a pharmaceutically acceptable salt thereof; and co-administering a therapeutically effective amount of at least one of: tetrahydrocannabinol (THC), and cannabidiol (CBI)). The method is provided, in one embodiment, wherein a duration of erection comprises at least one hour. In another embodiment, the duration of erection comprises at least three hours.

In yet a further embodiment, the duration of erection comprises at least five hours. In yet a further embodiment the duration of erection is extended for an extended period of time while the THC:CBD ratio treats the mental (PTSD) and physical (pain) symptoms of the patient that interfere with optimal sexual performance of the patient that the sildenafil alone cannot treat, creating a holistic healing approach to the sexual performance.

While the compositions and methods described herein are directed to the treatment of or prevention of erectile dysfunction (ED) in males, the methods and formulations may be used to treat female sexual dysfunction (BD) as well. These methods and formulations can be used in combination with or independently of other libido enhancing, FSD for ED treatment methods and compositions.

1. A pharmaceutical composition for treatment of erectile dysfunction, comprising:
a therapeutically effective amount of a phosphodiesterase-5 (PDE-5) inhibitor, or a pharmaceutically acceptable salt thereof; and
a therapeutically effective amount of at least one of:
tetrahydrocannabinol (THC), and
cannabidiol (CBD).

2. The pharmaceutical composition of claim 1, wherein the PDE-5 inhibitor comprises sildenafil and/or tadalafil.

3. The pharmaceutical composition of claim 1, wherein the CBD is extracted from cannabis indica.

4. The pharmaceutical composition of claim 1, comprising both THC and CBD, wherein the THC and CBD are provided in a 5:2 ratio (THC:CBD).

5. The pharmaceutical composition of claim 1, further comprising a pharmaceutically acceptable carrier.

6. The pharmaceutical composition of claim 1, comprising 0.01 mg-150 mg of the PDE-5 inhibitor.

7. The pharmaceutical composition of claim 6, comprising 0.1 mg-100 mg of the PDE-5 inhibitor.

8. The pharmaceutical composition of claim 1, comprising 0.01 mg-150 mg of THC.

9. The pharmaceutical composition of claim 8, comprising 0.1 mg-100 mg of THC.

10. The pharmaceutical composition of claim 1, comprising 0.01 mg-1500 mg of CBD.

11. The pharmaceutical composition of claim 10, comprising 0.1-1000 mg of CBD.

12. The pharmaceutical composition of claim 1, wherein PDE-5 inhibitor comprises sildenafil and/or tadalafil.

13. A pharmaceutical composition comprising a phosphodiesterase-5 (PDE-5) inhibitor and THC or CBD for the treatment of erectile dysfunction.

14. A method of eliciting or prolonging an erection in a patient, or both, comprising:
administering to the patient a therapeutically effective amount of a phosphodiesterase-5 (PDE-5) inhibitor, or a pharmaceutically acceptable salt thereof; and
co-administering a therapeutically effective amount of at least one of:
tetrahydrocannabinol (THC), and
cannabidiol (CBD).

15. The method of claim 14, wherein the PDE-5 inhibitor comprises sildenafil and/or tadalafil.

16. The method of claim 14, further comprising wherein both THC and CBD are administered to the patient.

17. The method of claim 14, wherein the THC and CBD are administered to the patient in a 5:2 ratio (THC:CBD).

18. (canceled)

19. The method of claim 14, wherein a duration of erection comprises at least one hour.

20. The method of claim 14, wherein a duration of erection comprises at least three hours.

21. The method of claim 14, wherein the PDE-5 inhibitor is administered prior to administration of the THC and/or the CBD.
